(19) United States
(12) Patent Application Publication
(10) Pub. No.: US 2017/0007201 A1
(43) Pub. Date: Jan. 12, 2017

(54) ULTRASONIC DIAGNOSTIC APPARATUS AND MEDICAL IMAGE PROCESSING APPARATUS

(71) Applicant: TOSHIBA MEDICAL SYSTEMS CORPORATION, Otawara-Shi (JP)

(72) Inventors: Yutaka KOBAYASHI, Nasushiobara (JP); Kazutoshi SADAMITSU, Otawara (JP); Jiro HIGUCHI, Otawara (JP); Masatoshi NISHINO, Otawara (JP); Norihisa KIKUCHI, Otawara (JP); Atsushi SUMI, Otawara (JP); Naoyuki NAKAZAWA, Otawara (JP); Atsushi NAKAI, Nasushiobara (JP); Cong YAO, Otawara (JP); Naoki YONEYAMA, Yaita (JP); Kazuo TEZUKA, Nasushiobara (JP); Yoshitaka MINE, Nasushiobara (JP); Masami TAKAHASHI, Nasushiobara (JP)

(73) Assignee: TOSHIBA MEDICAL SYSTEMS CORPORATION, Otawara-Shi (JP)

(21) Appl. No.: 15/133,957

(22) Filed: Apr. 20, 2016

(30) Foreign Application Priority Data
Jul. 10, 2015 (JP) 2015-138292

(51) Int. Cl. A61B 8/08; G06T 7/20; G06T 7/00; A61B 5/0452; A61B 8/00

(52) U.S. Cl. CPC A61B 8/0883; A61B 5/0452; A61B 8/483; A61B 8/4494; G06T 7/0016; G06T 7/204; G06T 2207/10136; G06T 2207/30048

(57) ABSTRACT

An ultrasonic diagnostic apparatus according to a present embodiment includes processing circuitry. The processing circuitry is configured to extract a cardiac valve from three-dimensional images of frames generated by controlling an ultrasonic probe to perform transmission and reception of ultrasonic waves. The processing circuitry is configured to calculate, as a prolapse gap, a gap between valve leaflets of the cardiac valve in a three-dimensional image of a specific frame among the frames. The processing circuitry is configured to display the prolapse gap on a display.

### CROSS-REFERENCE TO RELATED APPLICATION

This application is based upon and claims the benefit of priority from Japanese Patent Application No. 2015-138292, filed on Jul. 10, 2015, the entire contents of which are incorporated herein by reference.

### FIELD

An embodiment relates to an ultrasonic diagnostic apparatus and a medical image processing apparatus.

### BACKGROUND

Disorders relating to malfunction of cardiac valves are called valvular diseases. The cardiac valves include a mitral valve, an aortic valve, a tricuspid valve, and a pulmonary valve. A disorder of the mitral valve, among these cardiac valves, is increasing in particular. The mitral valve contains two valve leaflets called an anterior leaflet and a posterior leaflet.

Disorders which influence one of the valve leaflets of the mitral valve include mitral regurgitation (MR). The mitral regurgitation is a case in which the mitral valve does not close properly and causes regurgitation of blood.

The mitral regurgitation was conventionally treated by surgical operations, such as “valvuloplasty” which restores the cardiac valve, and “valve replacement” which replaces the entire cardiac valve. However, in these days, there are cases of applying a technique called Mitral Clip (registered trademark) to the mitral regurgitation. In this technique, a device is sent through a catheter to treat the valve. In this technique, it is possible for a patient to be treated without thoracotomy in consideration of physical strength and age of the patient.

It is difficult to apply the Mitral Clip if the mitral valve has a prolapse gap of 10 [mm] or more in an end-systole that is a period when the mitral valve should essentially be closed. It Is because it become impossible to appropriately clip the anterior leaflet and the posterior leaflet of the mitral valve, when the prolapse gap of the mitral valve in the end-systole is too large.

Because of this reason, it is essential to preoperatively measure the prolapse gap of the mitral valve in the end-systole in order to determine whether or not the Mitral Clip is applicable. It is reported that measurement results of the prolapse gap of the mitral valve in the end-systole based on 3D images are closer to an actual prolapse gap than measurement results based on 2D images.

However, measuring the prolapse gap of the mitral valve in the end-systole involves specifying operation by an operator, which places a burden on the operator and causes variation in measurement depending on the skill of the operator.

To solve the above-stated problem, an object of a present invention is to provide an ultrasonic diagnostic apparatus and a medical image processing apparatus capable of presenting information on a prolapse gap of the cardiac valve.

### BRIEF DESCRIPTION OF THE DRAWINGS

In accompanying drawings,

FIG. 1 is a schematic view showing a configuration of an ultrasonic diagnostic apparatus according to a first embodiment;

FIG. 2 is a block diagram showing the functions of the ultrasonic diagnostic apparatus according to the first embodiment;

FIG. 3 is a conceptual diagram showing tracking of an edge of a mitral valve;

FIGS. 4A to 4D are diagrams each showing gaps between valve leaflets of the mitral valve in an end-systole;

FIGS. 5A to 5C are diagrams to each explain a method for calculating the gaps between the valve leaflets of the mitral valve in the end-systole;

FIG. 6 is a diagram showing a graph that indicates a relationship between positions of the gaps and the gaps at the corresponding positions;

FIG. 7 is a diagram to explain a method for collecting prolapse gap elements;

FIG. 8 is a diagram showing a result of collecting the prolapse gap elements as a graph;

FIG. 9 is a flow chart showing operations of the ultrasonic diagnostic apparatus according to the first embodiment;

FIG. 10 is a schematic diagram showing a configuration of a medical image processing apparatus according to a second embodiment; FIG. 11 is a block diagram showing functions of the medical image processing apparatus according to the second embodiment; and

FIG. 12 is a diagram showing a relationship between an electrocardiographic waveform and X-ray irradiation.

### DETAILED DESCRIPTION

An ultrasonic diagnostic apparatus and a medical image processing apparatus according to the present embodiment will be described with reference to the accompanying drawings.

The ultrasonic diagnostic apparatus according to the present embodiment includes processing circuitry. The processing circuitry is configured to extract a cardiac valve from three-dimensional images of frames generated by controlling an ultrasonic probe to perform transmission and reception of ultrasonic waves. The processing circuitry is configured to calculate, as a prolapse gap, a gap between valve leaflets of the cardiac valve in a three-dimensional image of a specific frame among the frames. The processing circuitry is configured to display the prolapse gap on a display.

### 1. First Embodiment

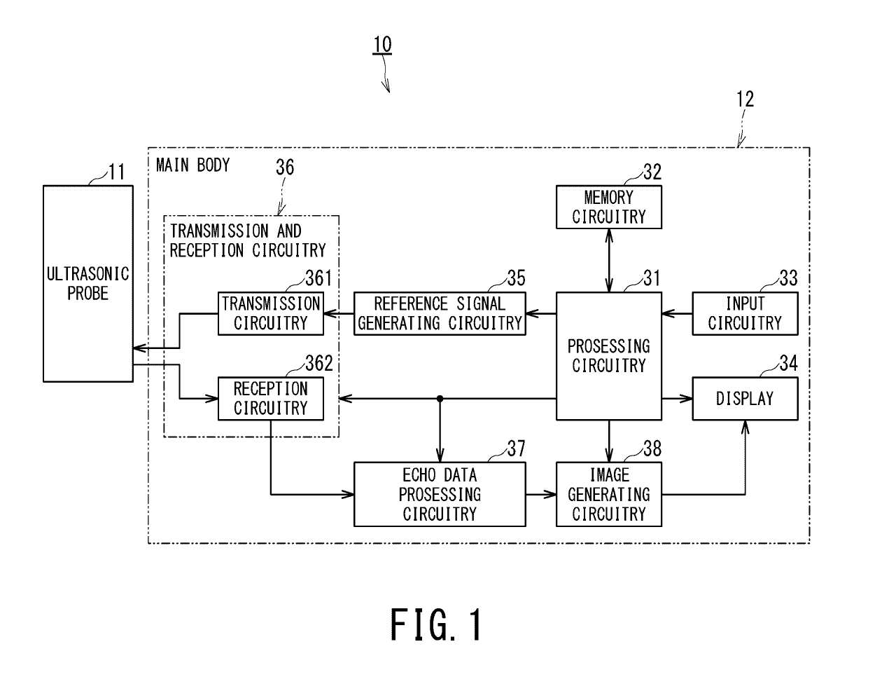

FIG. 1 is a schematic view showing a configuration of an ultrasonic diagnostic apparatus according to a first embodiment.

FIG. 1 shows an ultrasonic diagnostic apparatus 10 according to the first embodiment. The ultrasonic diagnostic apparatus 10 includes an ultrasonic probe 11 and a main body 12.

The ultrasonic probe 11 transmits and receives ultrasonic waves to/from an object. The ultrasonic probe 11 is configured to transmit and receive ultrasonic waves to/from an object with a front face of the probe in contact with a surface of the object. The ultrasonic probe 11 has (M pieces of) microscopic ultrasonic transducers arrayed in one dimension or two dimensions in a tip portion of the probe. The ultrasonic transducers are electroacoustic transduction elements having functions to convert an electrical pulse into an ultrasonic pulse (transmission ultrasonic wave) during transmission, or to convert an ultrasonic reflected wave (reception ultrasonic wave) into an electrical signal (reception signal) during reception.

The ultrasonic probe 11, which is configured to be small and lightweight, is connected to the main body 12 through a cable. The ultrasonic probe 11 includes a configuration to meet a sector scanning, a linear scanning mode, or a convex scanning. The ultrasonic probe 11 of multiple types of probes arbitrarily selected in accordance with a diagnostic region.

The main body 12 includes processing circuitry 31, memory circuitry (storage unit) 32, input circuitry (input unit) 33, a display (display unit) 34, reference signal generating circuitry 35, transmission and reception circuitry 36, echo data processing circuitry 37, and image generating circuitry 38.

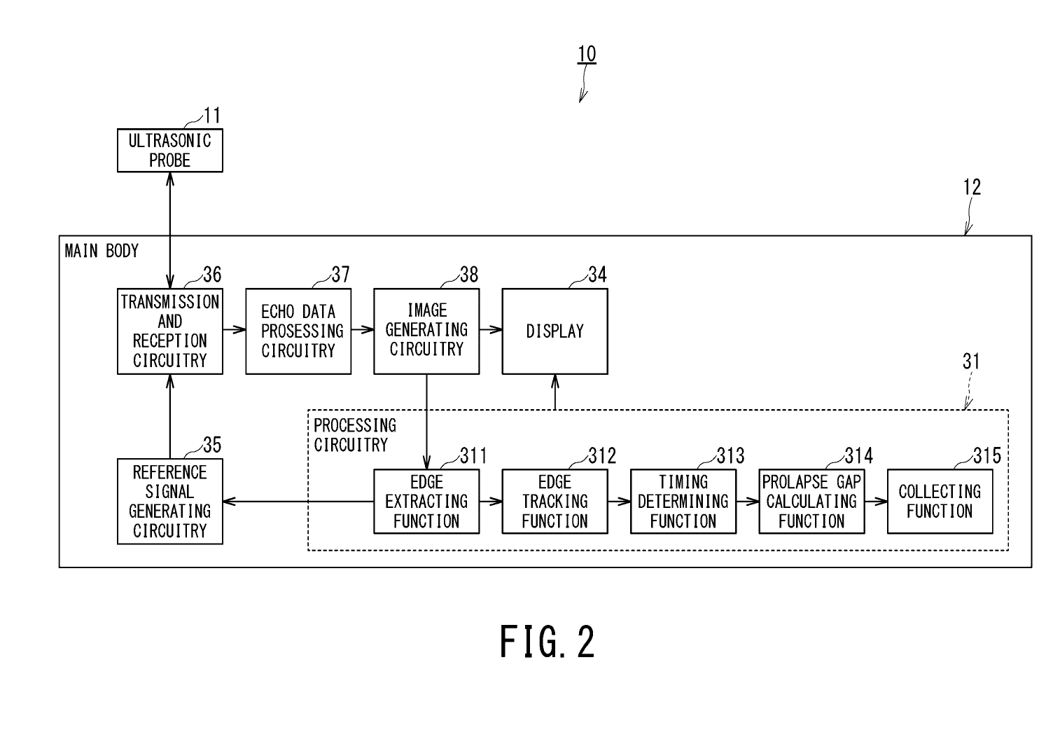

The processing circuitry 31 means any one of dedicated or general central processing unit (CPU) and a micro processor unit (MPU), an application specific integrated circuit (ASIC), and a programmable logic device. The programmable logic device may be, for example, any one of a simple programmable logic device (SPLD), a complex programmable logic device (CPLD), a field programmable gate array (FPGA) and the like. The processing circuitry 31 reads programs stored in the memory circuitry 32 or directly implemented in the processing circuitry 31, executes these programs, and performs the following functions 311 to 315 shown in FIG. 2.

The processing circuitry 31 may be a single processing circuit or a combination of multiple processing circuits. In the latter case, the memory circuitry 32 includes multiple memory circuits each storing an element of a program, each of the multiple memory circuits is provided for each of the multiple circuits. Alternatively, the memory circuitry 32 includes a single memory circuit storing the program, the single memory circuit is provided for the multiple circuits.

The memory circuitry 32 is configured by a semiconductor memory device such as a random access memory (RAM) and a flash memory, a hard disk, an optical disk, or the like. The memory circuitry 32 may be configured by a removable medium, such as a universal serial bus (USB) memory and a digital video disk (DVD). The memory circuitry 32 stores various processing programs (including application programs as well as an operating system (OS)) used in the processing circuitry 31, data necessary for execution of the programs, and medical images. The OS may also include a graphical user interface (GUI) which allows heavy use of graphics to display information on the display 34 for an operator and allow basic operations to be performed with the input circuitry 33.

The input circuitry 33 is configured to receive a signal input from an input device, such as a pointing device (such as a mouse) and a keyboard operable by the operator. In the present embodiment, the input device itself is included in the input circuitry 33. When the input device is operated by the operator, the input circuitry 33 generates an input signal corresponding to the performed operation and outputs the generated input signal to the processing circuitry 31. The main body 12 may include a touch panel having an input device integrated with the display 34.

The display 34 is configured by a general display output apparatus, such as a liquid crystal display and an organic light emitting diode (OLED) display. The display 34 displays image data generated by the image generating circuitry 38 under the control of the processing circuitry 31.

The reference signal generating circuitry 35 generates, for the transmission and reception circuitry 36, continuous waves or rectangular waves in response to a control signal from the processing circuitry 31. For example, the continuous waves or the rectangular waves have a frequency substantially equal to a center frequency of an ultrasonic pulse.

The transmission and reception circuitry 36 causes the ultrasonic probe 11 to perform transmission and reception in response to a control signal from the processing circuitry 31. The transmission and reception circuitry 36 includes transmission circuitry 361 that generates a driving signal for emitting transmission ultrasonic waves from the ultrasonic probe 11. The transmission and reception circuitry 36 also includes reception circuitry 362 that performs phasing and adding of reception signals from the ultrasonic probe 11.

The transmission circuitry 361 includes a rate pulse generator, a transmission delay circuitry, and a pulser, which are not shown. The rate pulse generator generates a rate pulse that determines a repeating cycle of a transmission ultrasonic wave by dividing the continuous wave or rectangular wave supplied from the reference signal generating circuitry 35. The rate pulse generator then supplies the generated rate pulse to the transmission delay circuitry. The transmission delay circuitry includes independent delay circuitries (M channels), the number of which is equal to the number of the transducers used for transmission. A delay time for converging transmission ultrasonic waves at a predetermined depth and a delay time for emitting transmission ultrasonic waves in a predetermined direction are given to the rate pulse to attain a thin beam width for transmission, and the rate pulse is supplied to the pulser. The pulser includes independent drive circuitries of M channels, and generates driving pulses for driving the transducers incorporated in the ultrasonic probe 11, based on the rate pulse.

The reception circuitry 362 of the transmission and reception circuitry 36 includes a preamplifier, analog to digital (A/D) conversion circuitry, reception delay circuitry, and an adder circuitry, which are not shown. The preamplifier includes M channels to amplify fine signals, which were converted into electric reception signals by the transducers, so as to secure sufficient signal to noise (S/N). The reception signals of M channels amplified to a predetermined magnitude in the preamplifier are converted into digital signals in the A/D conversion circuitry, and are sent to the reception delay circuitry. The reception delay circuitry imparts a convergence delay time and a deflection delay time to respective reception signals of M channels output from the A/D conversion circuitry. The convergence delay time is the time for converging ultrasonic reflected waves from a predetermined depth, while the deflection delay time is the time for setting reception directivity in a prescribed direction. The adder circuitry performs phasing and adding of the reception signals from the reception delay circuitry (phase-matching and adding of the reception signals obtained from a predetermined direction).

The echo data processing circuitry 37 performs ultrasonic image generation processing on echo data from the reception circuitry 362 in response to a control signal from the processing circuitry 31. For example, the echo data processing circuitry 37 performs B mode processing such as logarithmic compression processing and envelope detection processing, and Doppler processing such as orthogonal detection processing and filtering processing.

The image generating circuitry 38 scan-converts the data input from the echo data processing circuitry 37 into ultrasonic image data with a scan converter in response to a control signal from the processing circuitry 31. The image generating circuitry 38 then displays on the display 34 an ultrasonic image based on the ultrasonic image data. For example, the ultrasonic image is a B mode image or a color Doppler image.

A description is now given of functions of the ultrasonic diagnostic apparatus 10 according to the first embodiment.

FIG. 2 is a block diagram showing the functions of the ultrasonic diagnostic apparatus 10 according to the first embodiment.

When the processing circuitry 31 executes a program, the ultrasonic diagnostic apparatus 10 functions as an edge extracting function 311, an edge tracking function 312, a timing determining function 313, a prolapse gap calculating function 314, and a collecting function 315. Although a case where the functions 311 to 315 function as software will be described as an example, some or all of these functions 311 to 315 may each be provided in the ultrasonic diagnostic apparatus 10 as hardware such as a circuitry.

The edge extracting function 311 is configured to control operation of the ultrasonic probe 11 through the reference signal generating circuitry 35 so as to start 4D scan with B mode, and to extract an edge of a cardiac valve (valve ring) based on 3D images of frames generated by the image generating circuitry 38. The cardiac valve includes a mitral valve, an aortic valve, a tricuspid valve, and a pulmonary valve. Although a case of extracting the mitral valve will be described hereinafter as an example, the present invention is not limited thereto.

The edge tracking (chasing) function 312 is configured to track an edge of the mitral valve extracted by the edge extracting function 311, based on 3D images of frames generated by the image generating circuitry 38. In tracking the mitral valve, the edge tracking function 312 may apply a pattern matching technique to the edge of the mitral valve, as in the case of tracking a cardiac wall with a conventional wall motion tracking (WMT) technology.

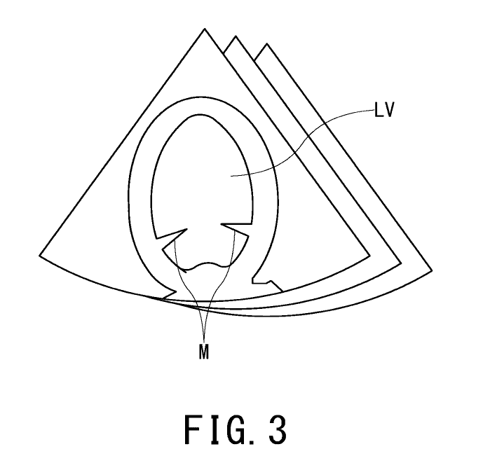

FIG. 3 is a conceptual diagram showing tracking of the edge of the mitral valve.

FIG. 3 shows 3D images of frames including a left ventricle (LV). In the technique of tracking the edge of a mitral valve M, a template image is set at the edge of the mitral valve M which is a portion subjected to motion analysis on a 3D image of a start frame. A position of the edge of the mitral valve M in a 3D image of a next frame is estimated by searching a region in the 3D image of the next frame, the region being best matched in a speckle pattern with the template image. By repeating this estimation processing between 3D images of consecutive two frames, the edge of the mitral valve M which changes over time is tracked.

With reference again to FIG. 2, the timing determining function 313 is configured to determine a timing that the mitral valve should be closed, such as an end-systole. The timing determining function 313 determines the end-systole based on an electrocardiogram (ECG) signal. The timing determining function 313 may calculates prolapse gaps between the valve leaflets of the cardiac valve in each of the three-dimensional images of frames, and may determine a frame having a maximum prolapse gap, among the prolapse gaps relating to the frames, as a frame in the end-systole.

The end-systole is preferably a time phase in which an absolute value of cardiac muscle velocity is the smallest in a specified period set between an S wave time phase and an E wave time phase based on an electrocardiogram signal. An end-systole determination method is disclosed, for example, Japanese Patent Laid-open No. 2005-342006.

The prolapse gap calculating function 314 is configured to calculate a prolapse gap based on a gap between the valve leaflets of the mitral valve in the end-systole, based on a 3D image of the frame corresponding to the end-systole determined by the timing determining function 313. The prolapse gap calculating function 314 may include a function to display a prolapse gap between the valve leaflets of the mitral valve in the end-systole on the display 34.

As a first example, the prolapse gap calculating function 314 calculates the prolapse gap as one gap between one point on an anterior leaflet and one point on a posterior leaflet of the mitral valve in the end-systole.

As a second example, the prolapse gap calculating function 314 calculates gaps each between a point on the anterior leaflet and a point on the posterior leaflet of the mitral valve in the end-systole, and uses a representative value of these gaps as the prolapse gap between the valve leaflets. Examples of the representative value include an average value and a maximum value in the gaps.

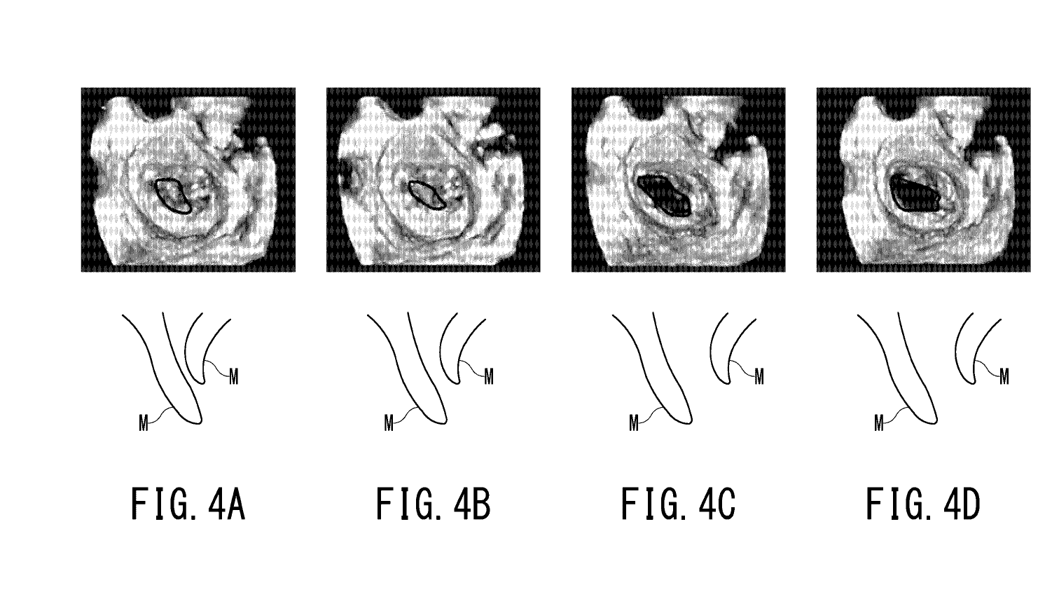

FIGS. 4A to 4D are diagrams each showing the gaps between the valve leaflets of the mitral valve in the end-systole.

In each of FIGS. 4A to 4D, an upper row shows a mitral valve as viewed from the top (left atrium), and a lower row shows the mitral valve as viewed from the side.

As shown in the upper row in each of FIGS. 4A and 4B, when the mitral valve is viewed from the top (left atrium) in a timing of the end-systole, there seems to be no gap between the valve leaflets. However, as shown in the lower row in each of FIGS. 4A and 4B, when the mitral valve is viewed from the side in the timing of the end-systole, a gap is present between the valve leaflets. In this manner, it is preferred to calculate the prolapse gap based on the gap(s) between the valve leaflets in the end-systole on three-dimensional images.

In contrast, with the timing of the end-systole shown in each of FIGS. 4C and 4D, the gap is present between the valve leaflets whether the mitral valve is viewed from the top or from the side.

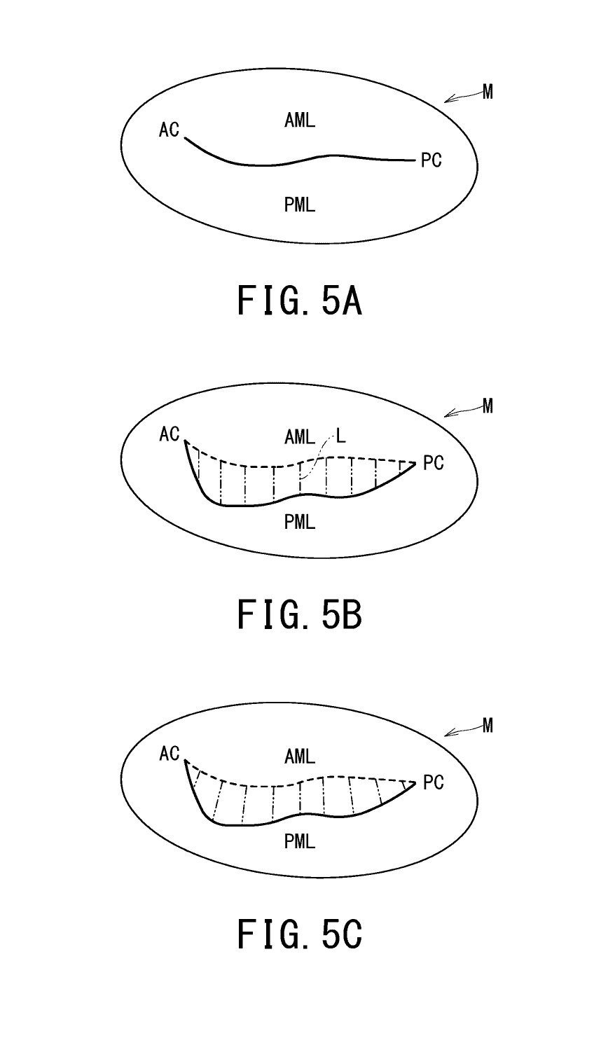

FIGS. 5A to 5C are diagrams to each explain a method for calculating the gaps between the valve leaflets of the mitral valve in the end-systole.

FIG. 5A shows a state of a normal mitral valve in the end-systole. FIGS. 5B and 5C show a state of mitral regurgitation.

As shown in FIG. 5B, a gap (line segment L) between a middle point of the edge of an anterior leaflet AML and a middle point on the edge of a posterior leaflet PML is calculated as the prolapse gap between the valve leaflets in the end-systole. The anterior leaflet AML and the posterior leaflet PML are positioned between an anterior commissure AC and a posterior commissure PC.

Alternatively, the line segment L and other line segments parallel thereto are obtained between the edge of the anterior leaflet AML and the edge of the posterior leaflet PML, and respective lengths of the segments are calculated as gaps. A representative value of these lengths, for example, a maximum gap of the gaps, is defined as the prolapse gap between the valve leaflets.

As shown in FIG. 5C, line segments are obtained by connecting points on the edge of the anterior leaflet AML to points on the edge of the posterior leaflet PML. The respective points are provided at equal intervals from the anterior commissure AC (or posterior commissure PC), and respective lengths of the line segments are calculated as gaps. A representative value of these lengths, for example, a maximum gap of the gaps, is defined as the prolapse gap between the valve leaflets.

With reference again to FIG. 2, the prolapse gap calculating function 314 can also calculate a position of the calculated gap of the mitral valve in the end-systole.

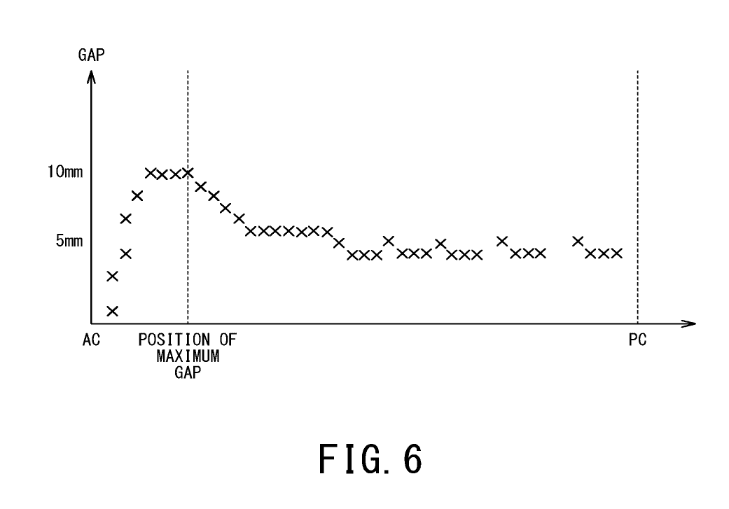

FIG. 6 is a diagram showing a graph that indicates a relationship between positions of the gaps and the gaps at the corresponding positions.

FIG. 6 shows a graph that plots the gaps at the corresponding positions of the gaps calculated in FIG. 5B or 5C. As shown in FIG. 6, a maximum gap of the gaps is present on the side close to the anterior commissure AC.

Unlike the mitral valve, other cardiac valves including an aortic valve, a tricuspid valve, and a pulmonary valve are each made up of three valve leaflets. In the case of calculating prolapse gaps between the valve leaflets of the aortic valve, the tricuspid valve, and the pulmonary valve, a position of one gap and a position of a maximum gap of gaps are each calculated between first and second valve leaflets, between second and third valve leaflets, and between third and first valve leaflets.

With reference again to FIG. 2, the collecting function 315 has a function which collects multiple prolapse gaps each relating to a heartbeat, and adopts the prolapse gaps as prolapse gap elements, the prolapse gaps being calculated by the prolapse gap calculating function 314. The collecting function 315 has a function which calculates one prolapse gap relating to the heartbeats on the basis of the prolapse gap elements. The collecting function 315 adopts, as the prolapse gap relating to the heartbeats, a representative value such as a maximum value or an average value of the prolapse gap elements.

Alternatively, the collecting function 315 has a function which averages gaps relating to the heartbeats and to the positions, the gaps being calculated by the prolapse gap calculating function 314. The collecting function 315 has a function which adopts, as a prolapse gap relating to the heartbeats, a representative value such as a maximum value or an average value of the averaged gaps of the corresponding positions.

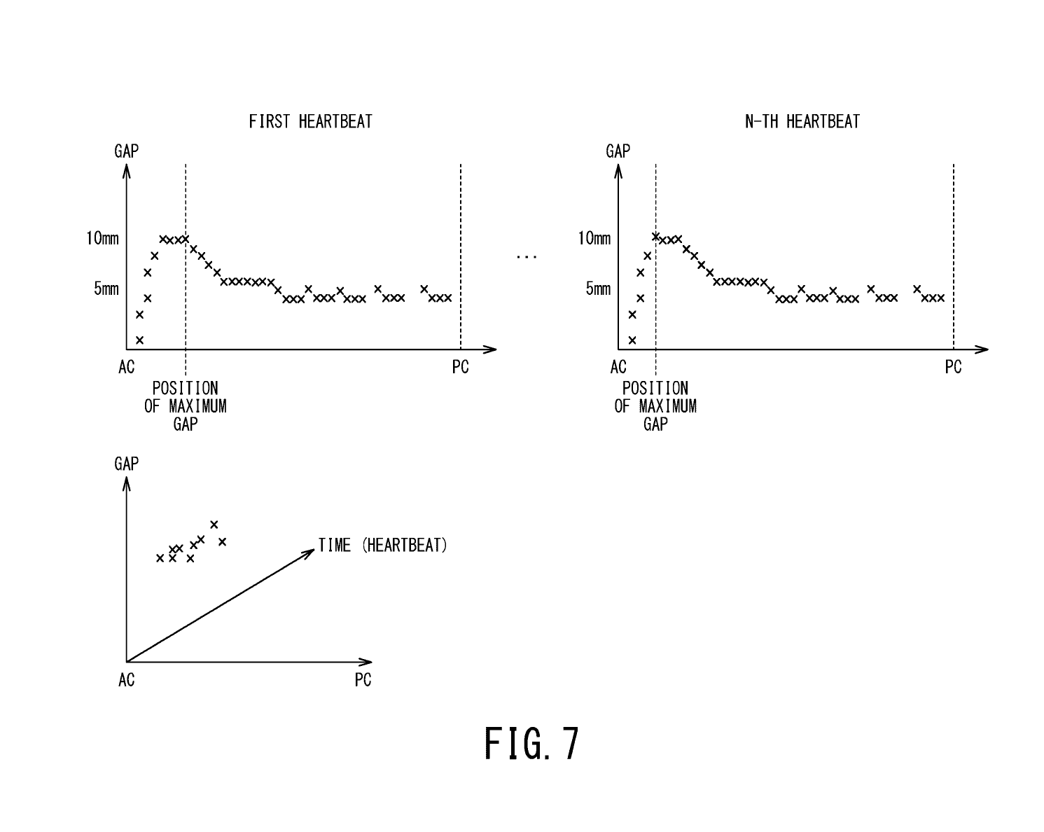

FIG. 7 is a diagram to explain a method for collecting the prolapse gap elements. In FIG. 7, an upper row shows two graphs (a first heartbeat to an N-th heartbeat) each indicating a relationship between positions of the gaps and the gaps during heartbeats. A lower row of FIG. 7 shows plots each indicating a relationship between a position of the prolapse gap element, time (heartbeat), and a gap of the prolapse gap element.

According to the lower row of FIG. 7, the position of the prolapse gap element changes in accordance with change in heartbeat.

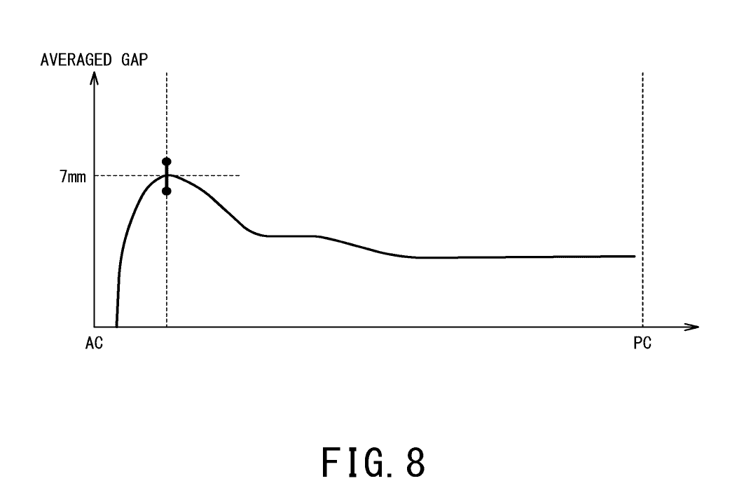

FIG. 8 is a diagram showing a result of collecting the prolapse gap elements as a graph.

FIG. 8 shows a curved line including multiple averaged gaps obtained by averaging the gaps relating to heartbeats with respect to each position. A maximum value of the curved line is calculated as one prolapse gap relating to the heartbeats. As shown in FIG. 8, a line (thick line between two points) representing variation (standard deviation or distribution) in gaps corresponding to the position of the maximum value of the curved line is shown at the position.

The curved line shown in FIG. 8 and/or text information indicating the position of one prolapse gap relating to heartbeats, one prolapse gap value relating to the heartbeats, and a value representative of deviation may be displayed, and this enables the operator to visually recognize one prolapse gap relating to the heartbeats.

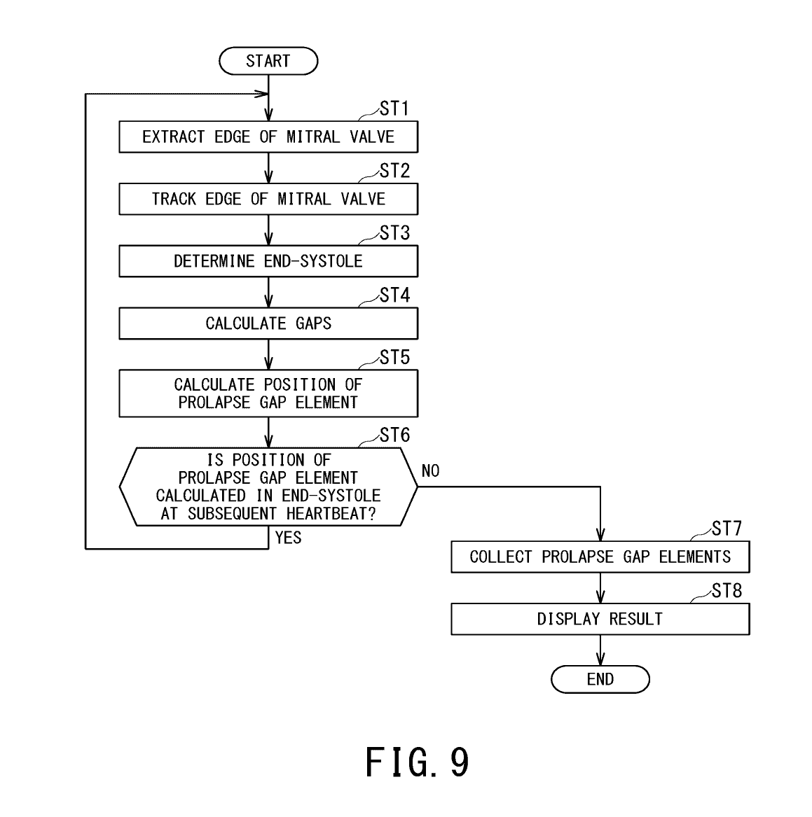

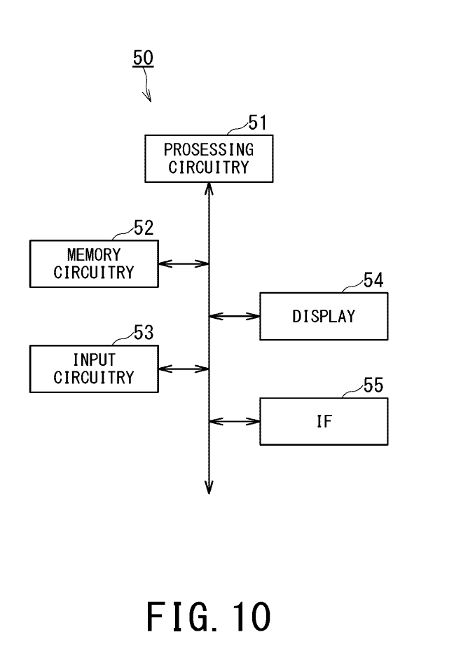

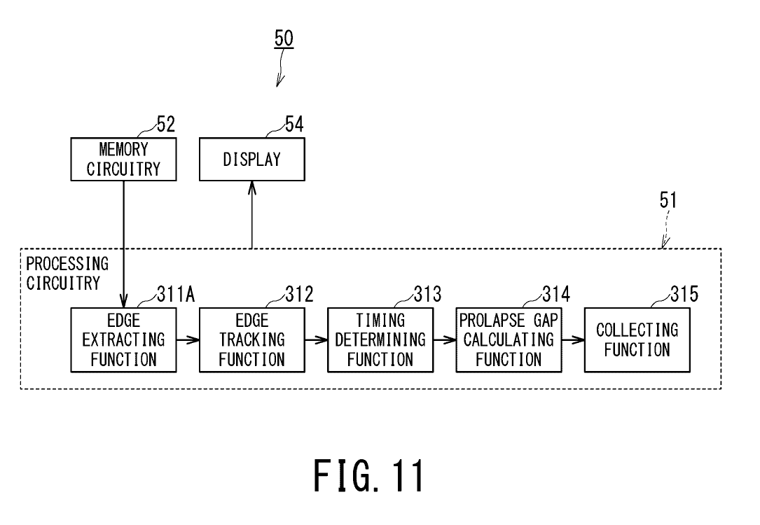

Next, operation of the ultrasonic diagnostic apparatus 10 will be described with reference to FIGS. 1 and 9.

FIG. 9 is a flow chart showing operations of the ultrasonic diagnostic apparatus 10 according to the first embodiment.

At a certain heat beat, the ultrasonic diagnostic apparatus 10 controls operation of the ultrasonic probe 11 through the reference signal generating circuitry 35 to start 4D scan with B mode, and extracts an edge of a mitral valve based on 3D images generated by the image generating circuitry 38 (step ST1). The ultrasonic diagnostic apparatus 10 tracks the edge of the mitral valve extracted in step ST1 based on 3D images of frames generated by the image generating circuitry 38 (step ST2).

The ultrasonic diagnostic apparatus 10 determines a timing that the mitral valve should close, for example, an end-systole (step ST3). The ultrasonic diagnostic apparatus 10 calculates gaps each between a point on the anterior leaflet and a point on the posterior leaflet of the mitral valve in the end-systole, based on a 3D image of the frame corresponding to the end-systole determined in step ST3 (step ST4).

The ultrasonic diagnostic apparatus 10 calculates a maximum value of the gaps calculated in step ST4 as a prolapse gap element of the mitral valve in the end-systole, and calculates a position of the prolapse gap element (step ST5).

The ultrasonic diagnostic apparatus 10 determines whether or not to calculate a position of a prolapse gap in the end-systole at a subsequent heartbeat (step ST6). When YES is determined in step ST6, that is, when it is determined to calculate the position of the prolapse gap element in the end-systole in the subsequent heartbeat, the ultrasonic diagnostic apparatus 10 controls operation of the ultrasonic probe 11 through the reference signal generating circuitry 35 to start 4D scan with B mode in the subsequent heartbeat, and extracts the edge of the mitral valve based on 3D images generated by the image generating circuitry 38 (step ST1).

On the contrary, when NO is determined in step ST6, that is, when it is determined not to calculate the position of the prolapse gap element in the end-systole in the subsequent heartbeat, the ultrasonic diagnostic apparatus 10 collects the prolapse gap elements each relating to heartbeat, and calculates one prolapse gap relating to the heartbeats (step ST7). The ultrasonic diagnostic apparatus 10 displays on the display 34 the positions of the prolapse gap elements each relating to the heartbeat calculated in step ST5, and/or one prolapse gap relating to the heartbeats calculated in step ST7 (step ST8).

The ultrasonic diagnostic apparatus 10 according to the first embodiment is able to provide the operator with a precise and accurate prolapse gap of the mitral valve in the end-systole. Furthermore, the ultrasonic diagnostic apparatus 10 according to the first embodiment is able to present the operator with one prolapse gap calculated from not only a prolapse gap relating to one heartbeat but also one prolapse gap calculated from the gaps each relating to the heartbeat. In this manner, the ultrasonic diagnostic apparatus 10 makes it possible to provide the operator with a more precise and accurate prolapse gap of the mitral valve in the end-systole.

In this manner, prior to application of the Mitral Clip technique which clips the valve leaflets of the cardiac valve, the ultrasonic diagnostic apparatus 10 according to the first embodiment is able to present information on the prolapse gap of the mitral valve, which is important in determining whether or not the Mitral Clip is applicable and in determining the number of clips when the Mitral Clip is applicable.

### 2. Second Embodiment

FIG. 10 is a schematic diagram showing a configuration of a medical image processing apparatus according to a second embodiment.

FIG. 10 shows a medical image processing apparatus 50 according to the second embodiment.

For example, the medical image processing apparatus 50 is a dedicated or general-purpose computer. For example, the function of the medical image processing apparatus 50 may be included in PCs (workstations) which perform image processing on medical images, medical image management apparatuses (servers) which store and manage medical images, or other apparatuses.

Hereinafter, a case where the medical image processing apparatus 50 is a dedicated or general-purpose computer will be described as an example.

The medical image processing apparatus 50 includes processing circuitry 51, memory circuitry (storage unit) 52, input circuitry (input unit) 53, a display (display unit) 54, and an IF (communication control circuitry) 55.

The processing circuitry 51 is similar in configuration to the processing circuitry 31 shown in FIG. 1. The memory circuitry 52 is similar in configuration to the memory circuitry 32 shown in FIG. 1. The input circuitry 53 is similar in configuration to the input circuitry 33 shown in FIG. 1. The display 54 is similar in configuration to the display 34 shown in FIG. 1.

The interface (IF) 54 performs communication operation with outside apparatuses based on a prescribed telecommunications protocol. When the medical image processing apparatus 50 is provided on a network, the IF 54 performs information exchange with the outside apparatuses on the network. For example, the IF 54 performs communication operation with outside apparatuses. The communication operation includes: receiving data obtained by imaging operation by a medical diagnostic imaging apparatus (not shown) such as an MRI apparatus, from the medical diagnostic imaging apparatus, a medical image management apparatus (not shown), or the like; and transmitting data generated by the medical image processing apparatus 50 to the medical image management apparatus or a diagnostic reading terminal (not shown).

A description is now given of functions of the medical image processing apparatus 50 according to the second embodiment.

FIG. 11 is a block diagram showing the functions of the medical image processing apparatus 50 according to the second embodiment.

When the processing circuitry 51 executes a program, the medical image processing apparatus 50 functions as an edge extracting function 311A, an edge tracking function 312, a timing determining function 313, a prolapse gap calculating function 314, and a collecting function 315. Although a case where the functions 311A to 315 function as software will be described as an example, some or all of these function 311A to 315 may each be provided in the medical image processing apparatus 50 as hardware such as a circuitry.

In FIG. 11, functions identical to those shown in FIG. 2 are designated by identical reference signs to omit a description thereof.

The edge extracting function 311A is configured to obtain (read) 3D images with B mode stored in the memory circuitry 52 and to extract an edge of a mitral valve (valve ring) based on the 3D images of frames. The cardiac valve includes a mitral valve, an aortic valve, a tricuspid valve, and a pulmonary valve. Although a case of extracting the mitral valve will be described hereinafter as an example, the present invention is not limited thereto.

The medical image processing apparatus 50 is able to calculate the prolapse gap of the cardiac valve from 3D images of B mode generated through 4D scan by the ultrasonic diagnostic apparatus 10 (shown in FIG. 1). Here, the medical image processing apparatus 50 is able to also calculate the prolapse gap of the cardiac valve from 3D images generated through 4D scan by an image generation apparatus other than the ultrasonic diagnostic apparatus 10, such as a X-ray computed tomography (CT) apparatus, by a method similar to the method for 3D images of B mode.

As the X-ray CT apparatus, a fifth-generation X-ray CT apparatus having a relatively high time resolution is preferably used. The fifth-generation X-ray CT apparatus is configured to generate an X-ray by irradiating anodes arranged in a semicircular shape with an electron beam, and to detect the X-ray with X-ray detectors arranged in an arc shape so as to face the anodes. In the case of utilizing the X-ray CT apparatus, 4D scan is performed only in a specific period based on an electrocardiogram signal so as to generate 3D images of heart phases in the specified period. This makes it possible to reduce X-ray exposure. The specific period is a whole period or a partial period of a cardiac systole including an end-systole in which a prolapse gap is calculated.

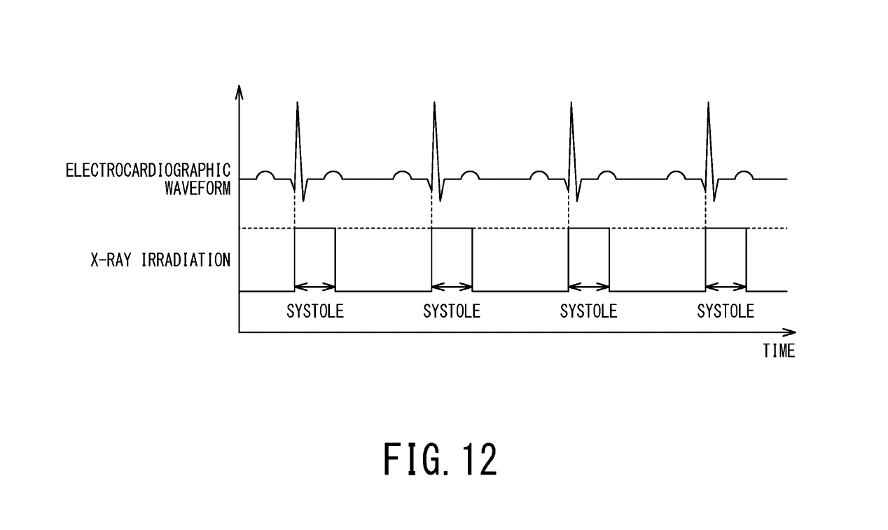

FIG. 12 is a diagram showing a relationship between an electrocardiographic waveform and X-ray irradiation.

In FIG. 12, an upper row shows an electrocardiographic waveform based on an electrocardiogram signal. In FIG. 12, a lower row shows repetition of X-ray irradiation (ON) and non-irradiation (OFF). As shown in the lower row of FIG. 12, X-ray irradiation, i.e., 4D scan, is performed in the whole period of the cardiac systole corresponding to the specific period. The X-ray irradiation in the systolic generates 3D images of heart phases in the systole. In this case, the generated 3D images of heart phases are equivalent to the 3D images of frames that are targets of edge tracking described before. A 3D image of a frame corresponding to the end-systole is identified out of the generated 3D images of heart phases by the method described before.

In a case where an image generation apparatus having a relatively low time resolution, such as a third-generation X-ray CT apparatus, is utilized, 3D images of heart phases may be generated from projection data over heartbeats. In that case, projection data in a different projection direction (view) is obtained for each heart phase from the projection data over the heartbeats. In this case, the generated 3D images of heart phases are equivalent to 3D images of frames that are target of edge tracking as described before. A 3D image of a frame corresponding to the end-systole is identified out of the generated 3D images of heart phases by the method described before.

The medical image processing apparatus 50 according to the second embodiment is able to provide the operator with a precise and accurate prolapse gap of the mitral valve in the end-systole. Furthermore, the medical image processing apparatus 50 according to the second embodiment is able to present the operator with one prolapse gap calculated from not only a prolapse gap relating to one heartbeat but also one prolapse gap calculated from the gaps on each relating to the heartbeat. In this manner, the medical image processing apparatus 50 makes it possible to provide the operator with a more precise and accurate prolapse gap of the mitral valve in the end-systole.

In this manner, prior to application of the Mitral Clip technique which clips the valve leaflets of the cardiac valve, the medical image processing apparatus 50 according to the second embodiment is able to present information on a prolapse gap of the mitral valve, which is important in determining whether or not the Mitral Clip is applicable and in determining the number of clips when the Mitral Clip is applicable.

According to the ultrasonic diagnostic apparatus and the medical image processing apparatus in at least one embodiment described in the foregoing, information on the prolapse gap of the cardiac valve is able to be presented.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus, comprising:
processing circuitry configured to
extract a cardiac valve from three-dimensional images of frames generated by controlling an ultrasonic probe to perform transmission and reception of ultrasonic waves,
calculate, as a prolapse gap, a gap between valve leaflets of the cardiac valve in a three-dimensional image of a specific frame among the frames, and
display the prolapse gap on a display.

2. The ultrasonic diagnostic apparatus according to claim 1, wherein
the processing circuitry is configured to define a frame corresponding to an end-systole as the specific frame.

3. The ultrasonic diagnostic apparatus according to claim 2, wherein
the processing circuitry is configured to determine the end-systole based on an electrocardiogram signal.

4. The ultrasonic diagnostic apparatus according to claim 2, wherein
the processing circuitry is configured to
calculate prolapse gaps between the valve leaflets of the cardiac valve with respect to the three-dimensional images of the frames, and
determine, as the end-systole, a frame having a maximum prolapse gap among the prolapse gaps relating to the frames.

5. The ultrasonic diagnostic apparatus according to claim 1, wherein
the processing circuitry is configured to calculate, as the prolapse gap, one gap between the valve leaflets of the cardiac valve in the three-dimensional image of the specific frame.

6. The ultrasonic diagnostic apparatus according to claim 1, wherein
the processing circuitry is configured to
calculate gaps of gap positions each between the valve leaflets of the cardiac valve in the three-dimensional image of the specific frame, and
calculate, as the prolapse gap, a maximum prolapse gap among the gaps.

7. The ultrasonic diagnostic apparatus according to claim 1, wherein
the processing circuitry is configured to extract the cardiac valve in the three-dimensional image of the specific frame by tracking an edge of the cardiac valve.

8. The ultrasonic diagnostic apparatus according to claim 1, wherein
the processing circuitry is configured to
calculate, as prolapse gap elements, gaps each between the valve leaflets of the cardiac valve in the three-dimensional image of the specific frame, the gaps each relating to a heartbeat, and
collect the prolapse gap elements each relating to the heartbeat to calculate the prolapse gap.

9. The ultrasonic diagnostic apparatus according to claim 8, wherein
the processing circuitry is configured to adopt, as the prolapse gap relating to the heartbeats, a representative value of the prolapse gap elements.

10. The ultrasonic diagnostic apparatus according to claim 9, wherein
the processing circuitry is configured to adopt the representative value as a maximum value.

11. The ultrasonic diagnostic apparatus according to claim 1, wherein
the processing circuitry is configured to
calculate gaps each between the valve leaflets of the cardiac valve in the three-dimensional image of the specific frame, the gaps each relating to a heartbeat and a gap position,
average the gaps with respect to each gap position, and
adopt, as the prolapse gap, a representative value of the averaged gaps of the corresponding gap positions.

12. The ultrasonic diagnostic apparatus according to claim 11, wherein
the processing circuitry is configured to adopt the representative value as a maximum value.

13. The ultrasonic diagnostic apparatus according to claim 1, wherein
the cardiac valve is a mitral valve.

14. A medical image processing apparatus, comprising:
processing circuitry configured to
obtain three-dimensional images of frames from a storage unit,
extract a cardiac valve from the three-dimensional images of the frames,
calculate, as a prolapse gap, a gap between valve leaflets of the cardiac valve in a three-dimensional image of a specific frame among the frames, and
display the prolapse gap on a display.
